(19) United States
(12) Patent Application Publication
(10) Pub. No.: US 2022/0379124 A1
(43) Pub. Date: Dec. 1, 2022

(54) Wirelessly Powered Stimulator

(71) Applicant: The Regents of the University of California, Oakland, CA (US)

(72) Inventors: Aydin Babakhani, Los Angeles, CA (US); Hongming Lyu, Los Angeles, CA (US)

(73) Assignee: The Regents of the University of California, Oakland, CA (US)

(21) Appl. No.: 17/753,930

(22) PCT Filed: Aug. 26, 2020
(86) PCT No.: PCT/US20/48001

§ 371 Date: Mar. 18, 2022

Related U.S. Application Data
(60) Provisional application No. 62/902,216, filed on Sep. 18, 2019.

(51) Int. Cl. A61N 1/378; H02J 50/27; H02J 50/12; A61N 1/36

(52) U.S. Cl. CPC A61N 1/3787; H02J 50/27; H02J 50/12; A61N 1/36192; H02J 2207/50

(57) ABSTRACT

Wirelessly powered implantable pulse generators (IPG) are described. In an embodiment, a wirelessly powered stimulator, includes an implantable pulse generator (IPG), including: an Rx antenna that receives a radio frequency (RF) signal from an external Tx antenna; a rectifier; an energy storage capacitor C_{STOR}, where the RF signal coupled to the Rx antenna is rectified by the rectifier to generate VDD and charges the C_{STOR}; a demodulator; an output voltage regulator that generates a stable voltage to activate the demodulator; and where the demodulator outputs a stimulation that releases the energy stored in the C_{STOR} on an electrode based on detecting amplitude modulation in the received RF signal; and a Tx antenna that generates the RF signal that wirelessly powers the IPG and that controls timing of output stimulations of the IPG, where amplitude modulation is applied to the RF signal to control the timing of the output stimulations.

### CROSS-REFERENCED APPLICATIONS

This application is a national stage of PCT Patent Application No. PCT/US2020/048001 entitled “Wirelessly Powered Stimulator” filed Aug. 26, 2020, which claims priority to U.S. Provisional application No. 62/902,216 filed on Sep. 18, 2019, entitled “Wirelessly Powered Stimulator”, the disclosures of which are included herein by reference in their entirety.

### STATEMENT OF FEDERALLY SPONSORED RESEARCH

This invention was made with government support under Grant Number 1533688, awarded by the National Science Foundation. The government has certain rights in the invention.

### FIELD OF THE INVENTION

The present invention generally relates to wirelessly powered implantable pulse generators (IPG).

### BACKGROUND OF THE INVENTION

Implantable pulse generators (IPGs) have solved various critical clinical problems and improved the quality of human life. Their applications can include chronic pain relief, motor function recovery for spinal cord injuries, the treatment of gastroesophageal reflux disease, cardiac pacemaking, and curing stress urinary incontinence, among various other applications. Conventional IPGs are bulky with the battery taking up most of the unit, and the necessary leads are prone to cause various complications.

### SUMMARY OF THE DISCLOSURE

Systems and methods for wirelessly powered stimulators in accordance with embodiments of the invention are disclosed. In one embodiment, a wirelessly powered stimulator, includes: an implantable pulse generator (IPG), including: an Rx antenna that receives a radio frequency (RF) signal from an external Tx antenna, a rectifier, an energy storage capacitor C_{STOR}, where the RF signal coupled to the Rx antenna is rectified by the rectifier to generate VDD and charges the C_{STOR}, a demodulator, an output voltage regulator that generates a stable voltage to activate the demodulator; and where the demodulator outputs a stimulation that releases the energy stored in the C_{STOR} on an electrode based on detecting amplitude modulation in the received RF signal, and a Tx antenna that generates the RF signal that wirelessly powers the IPG and that controls timing of output stimulations of the IPG, where amplitude modulation is applied to the RF signal to control the timing of the output stimulations.

In a further embodiment, the IPG further includes several reverse bias diodes that release energy from the C_{STOR} when the energy stored reaches an upper level threshold.

In a further embodiment again, the Rx antenna is at least one antenna selected from the group consisting of an inductor coil, a resonant coil, a dipole antenna, a monopole antenna, a patch antenna, a bow-tie antenna, a phased-array antenna, and a wire.

In still a further embodiment, the C_{STOR} is off-chip.

In a further embodiment still, the C_{STOR} is on-chip.

In a further embodiment again, the Rx antenna is off-chip.

In a further embodiment yet again, the Rx antenna is on-chip.

In yet a further embodiment, amplitude modulation includes detecting at least a threshold percentage reduction in power of the RF signal from the Tx antenna.

In still a further embodiment again, the IPG further includes a DC-block capacitor, C_{BCK}, that delivers the output stimulations for charge-neutralization.

In still a further embodiment again still, the IPG further includes a discharge resistor, R_{DIS}, that nulls the accumulated charge on the C_{BCK}.

In still a further embodiment yet again, the IPG is used for at least one application selected from the group consisting of neural stimulation, heart pacing, defibrillation, bladder stimulation and deep brain stimulation.

In yet still a further embodiment again, the output voltage regulator limits an amplitude of output stimulations within a specific range, where the output voltage regulator enables the demodulator when a supply voltage exceeds a lower tier, and where when the supply voltage exceeds a higher tier, enables a discharge path to rapidly discharge excess incident charge.

In still a further embodiment again, the amplitude modulation is applied to the RF signal to control at least one of a repetition rate and a duration of the output stimulation in an analog manner.

In still a further embodiment again, the demodulator replicates a timing of the amplitude modulation applied to the RF signal.

In still a further embodiment again, the demodulator includes three source follower replicas with a high end V_{H}, low end V_{L}, and transient envelop V_{ENV} of the RF signal and the V_{ENV} detection branch uses a small capacitor Cₛₘ and V_{H} and V_{L} are extracted on large capacitors with and without the AC input respectively.

In still a further embodiment again, an average of V_{H} and V_{L}, V_{M}, is obtained using a resistive divider and compared with V_{ENV} to reconstruct the timing of the amplitude modulation.

In still a further embodiment again, a recovered timing signal is sharpened by a buffer.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The description and claims will be more fully understood with reference to the following figures and data graphs, which are presented as exemplary embodiments of the invention and should not be construed as a complete recitation of the scope of the invention.

FIG. 1 illustrates an in vivo experiment in which an IPG is fully implanted and used to stimulate the animal's hind limb muscle in accordance with an embodiment of the invention.

FIG. 2A illustrates a circuitry overview, with the circuit architecture of an IPG in accordance with an embodiment of the invention.

FIG. 2B illustrates a schematic of the Tx coil in accordance with an embodiment of the invention.

FIG. 3 illustrates a circuit schematic of a demodulator in accordance with an embodiment of the invention.

FIG. 4 illustrates a circuit schematic of an output voltage regulator in accordance with an embodiment of the invention. In particular, FIGS. 4A and 4B illustrates setting the high and low bars of the output amplitude, respectively, and FIG. 4C generates the voltage reference in accordance with an embodiment of the invention.

FIG. 5 illustrates an overall current consumption of the IC and that of the individual blocks in accordance with an embodiment of the invention.

FIG. 6 illustrates a circuit model of an energy-harvesting frontend resonator in accordance with an embodiment of the invention.

FIG. 7A illustrates a 3D model of an implemented Rx coil in accordance with an embodiment of the invention.

FIG. 7B illustrates a picture of an as-fabricated PCB incorporating an Rx coil in accordance with an embodiment of the invention.

FIG. 8A illustrates a simplified model of an energy-harvesting frontend resonator in accordance with an embodiment of the invention.

FIG. 8B illustrates a circuit schematic of a Dickson rectifier in accordance with an embodiment of the invention.

FIG. 9A illustrates a 3-dB bandwidth and FIG. 9B illustrates normalized Q_{η} for different rectifier designs in accordance with an embodiment of the invention.

FIG. 10 illustrates a simulated dependence of R_{REC} and C_{REC} on I_{LOAD} in accordance with an embodiment of the invention.

FIG. 11 illustrates a resonant frequency drift in muscle medium in accordance with an embodiment of the invention.

FIG. 12 illustrates a co-design procedure for the Rx coil and the rectifier, which ensures optimal performance at a specific Med Radio band in accordance with an embodiment of the invention.

FIG. 13 illustrates a microscopic image of a fabricated IC in accordance with an embodiment of the invention.

FIG. 14 illustrates a picture of an as-fabricated IPG assembly in comparison with a U.S. dime in accordance with an embodiment of the invention.

FIG. 15A illustrates a picture of a Tx coil in accordance with an embodiment of the invention.

FIG. 15B illustrates the Tx coil's S11 according to measurement in accordance with an embodiment of the invention.

FIG. 16 illustrates an output voltage waveform of an IPG in response to a 6 μs notch, the inset shows the equivalent circuit model for the electrode in accordance with an embodiment of the invention.

FIG. 17 illustrates voltage (a, c) and the resulting current (b, d) waveforms for a 96.7 μs pulse and a 197.6 μs pulse, respectively. (e) Three cycles of 96.7 μs pulses at 10 Hz rate in accordance with an embodiment of the invention.

FIG. 18A illustrates a maximum-distance operations in the air and FIG. 18B illustrates through water with Tx power of 1 W in accordance with an embodiment of the invention.

FIG. 19 illustrates output waveforms of an IPG with the LED loading the output in accordance with an embodiment of the invention.

FIG. 20 illustrates (a) an animal experiment setup. The inset shows the implantation of the IPG in accordance with an embodiment of the invention. (B) illustrates a closer view of the implantation site where the skin is sutured covering the device in accordance with an embodiment of the invention.

FIG. 21A illustrates transient recording of the induced force in response to 16.7 and 96.7 μs pulses, and FIG. 21B illustrates the dependence of the induced force on the pulse width in accordance with an embodiment of the invention.

FIG. 22 illustrates simulated 10-g average SAR when the Tx coil is placed at a distance of 3 cm from a male right leg model in ANSYS in accordance with an embodiment of the invention.

FIG. 23 illustrates a table providing a comparison of recently published battery-less IPGs.

### DETAILED DESCRIPTION OF THE DRAWINGS

Turning now to the drawings, implantable pulse generators (IPGs) in accordance with various embodiments of the invention are illustrated. Many embodiments provide for achieving battery-less and leadless IPGs that can be directly implanted in the specific anatomical region.

Most stimulation devices function in either current or voltage modes. The current-controlled stimulation (CCS) provides precise current control irrelevant of the load impedance. However, because the stimulator needs to comply with the worst-case electrode/tissue impedance condition, the CCS renders the worse energy efficiency in most clinical settings. The voltage-controlled stimulation (VCS) regulates the stimulus in the voltage domain and renders an excellent energy efficiency. Due to this reason, most existing commercially available IPGs are based on VCS. A physician identifying the appropriate range of stimulus strength in advance and over time can eliminate the chance of overstimulation.

Wireless power transfer is a substitute for the battery that powers implantable medical devices (IMDs). Aside from far/mid-field coupling and ultrasonic transmission, the near-field inductive coupling is an attractive developing technology. The medical device radiocommunications (MedRadio) service, e.g., 401-406, 413-419, 426-432, 438-444, and 451-457 MHz, assigned by the federal communications commission has been used for the telemetry of IMDs. Unlike hundreds-MHz prior art that adopts on-chip coils, many embodiments of the IPG implement a miniaturized Rx coil on a PCB to minimize the cost. Also, in many embodiments of the IPG, a discrete energy storage capacitor is regardless used to be assembled with the integrated circuitry.

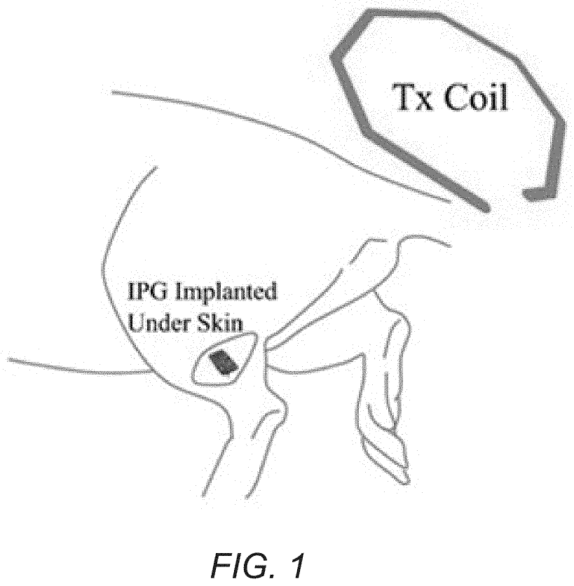

Accordingly, many embodiments provide a concise circuitry to realize an energy-efficient voltage-controlled IPG with a quiescent (while not stimulating) current consumption of 950 nA. In several embodiments, inductive coupling at a MedRadio band can achieve the wireless power link, where notches may be intentionally applied to precisely control the width and rate of the output pulses in an analog manner. In many embodiments, the energy-harvesting frontend circuitry takes account of the potential impacts of biological tissues. In many embodiments, the finalized assembly features an overall dimension of 4.6 mm×7 mm with the Rx coil size of 4.5 mm×3.6 mm. The potential use of an IPG in accordance with an embodiment of the invention in correcting the foot drop was verified in an in vivo study in which the IPG was implanted at the hindlimb muscle (Tibialis Anterior) belly of an anesthetized rat under the skin, as illustrated in FIG. 1 in accordance with an embodiment of the invention. In many embodiments, isolated contractions of the ankle joint were induced with controllable rates and forces.

Described are circuit implementations of IPGs in with a focus on the design tradeoffs in the energy-harvesting frontend circuitry in accordance with several embodiments of the invention. Furthermore, a discussion of the benchtop measurement and in vivo experiment results are provided.

### Circuit Implementation

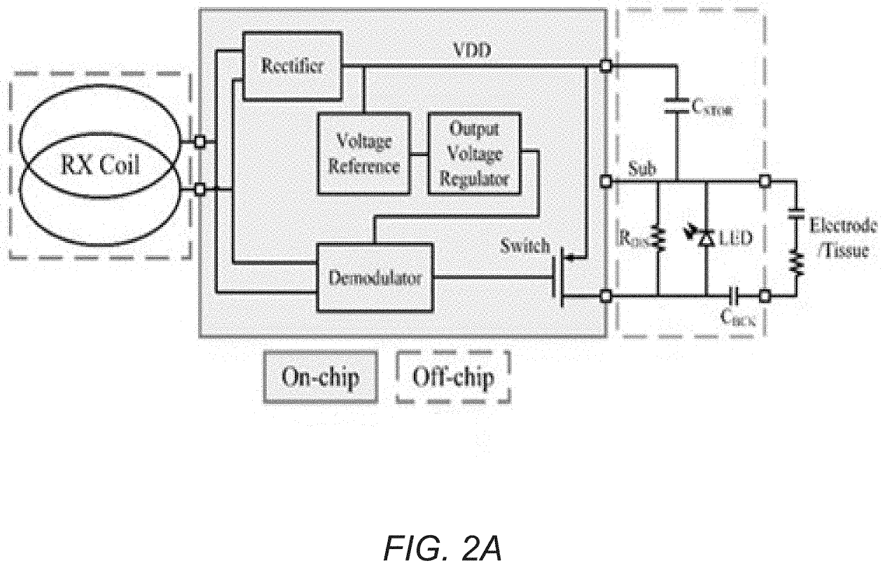

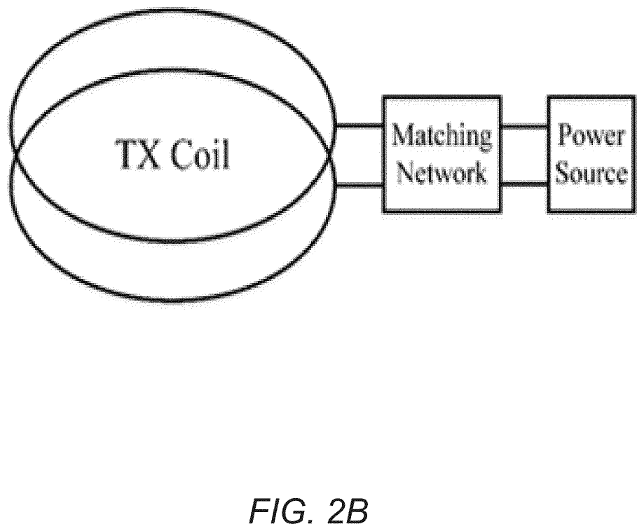

A systematic architecture of an IPG in accordance with an embodiment of the invention is shown in FIG. 2A. In many embodiments, the magnetic field coupled to the Rx coil can be rectified to generate VDD and charges an energy storage capacitor, C_{STOR}. In several embodiments, notches (e.g., RF power is reduced to a percentage of the RF power during harvest) can be intentionally applied in the Tx signal which precisely controls the timing of the output stimulations as their repetitions. The notch-based modulation scheme can eliminate any complex telemetry and minimizes the power consumption. In many embodiments, as the notches only constitute a negligible portion of the Tx power, they do not degrade the efficiency of the power transfer link. In many embodiments, a VCS scheme may be adopted for better energy-efficiency, in which VDD node can be directly applied to the electrode/tissue with a controllable pulse width. In many embodiments, in replacement of a low-dropout (LDO), a simplified output voltage regulator may be used to limit the amplitude of the output stimulations within a specific range, which may further reduce the static power consumption. In many embodiments, the regulator may enable the notch-demodulation block only when the supply voltage exceeds the lower tier. On the contrary, when the supply voltage exceeds the higher tier, a discharge path may be enabled to rapidly discharge the excess incident charge. The stimulations can be delivered through a DC-block capacitor, C_{BCK}, for charge-neutralization. In several embodiments, a discharge resistor, R_{DIS}, nulls the accumulated charge on C_{BCK}. A light-emitting diode (LED) can be optionally included at the output. Although FIG. 2A illustrates a particular circuit architecture of an IPG, any of a variety of circuit architectures may be utilized as appropriate to the requirements of specific applications in accordance with embodiments of the invention.

In many embodiments, an IPG can be wirelessly powered and controlled by a custom Tx coil with the diameter of approximately 3 cm, as illustrated in FIG. 2B in accordance with an embodiment of the invention. In certain embodiments, a matching network ensures the impedance matching at approximately 430 MHz, the resonant frequency of the Rx energy-harvesting frontend. Although FIG. 2B illustrates a particular schematic of a Tx coil, any of a variety of architectures may be utilized as appropriate to the requirements of specific applications in accordance with embodiments of the invention.

### Demodulator

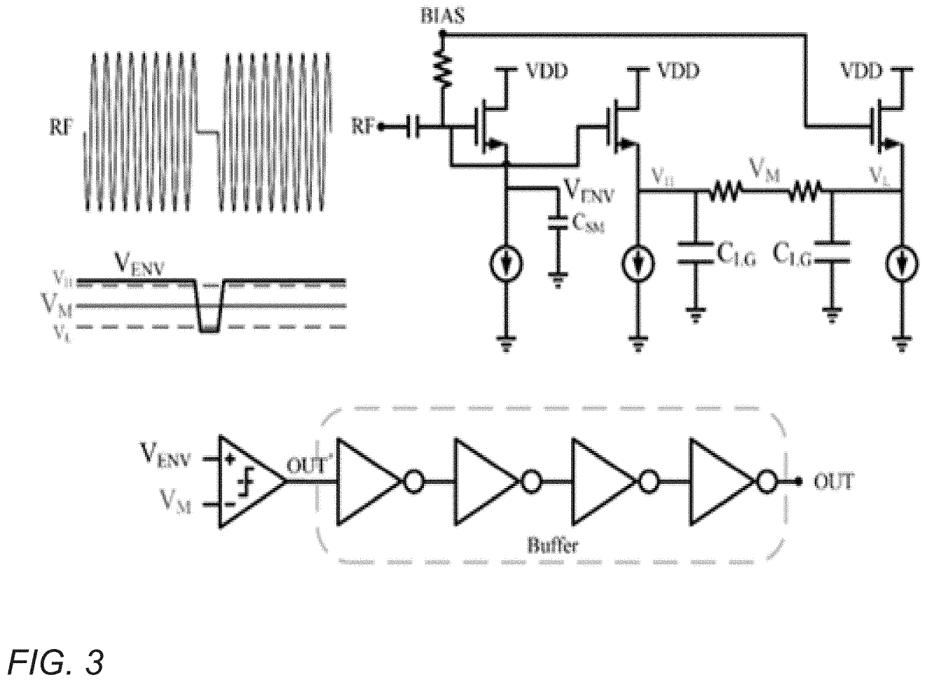

In many embodiments, a demodulator block can be responsible for replicating the timing of the notch, as shown in FIG. 3 in accordance with an embodiment of the invention. The conceptual waveforms of the incident signal 310 and the voltage of the critical nodes 320 in the demodulator are illustrated in FIG. 3. In many embodiments, the circuit can include three source follower replicas. The high end, low end, and transient envelope of the signal are denoted as V_{H}, V_{L}, and V_{ENV}, respectively. The V_{ENV} detection branch may use a relatively small capacitor, C_{SM}, while V_{H} and V_{L} can be extracted on larger capacitors with and without the AC input, respectively. Because of the nonlinearity of the transistors' transfer characteristics, an AC swing applied on a constant gate bias may generate a larger source voltage. The average of V_{H} and V_{L}, V_{M}, can be obtained through a resistive divider, which can thereafter be compared with V_{ENV} to reconstruct the timing of the notch. C_{SM} and C_{LG} can be selected to be 100 fF and 36 pF, respectively. As C_{SM}<<C_{LG}, V_{M} can be considered as constant so that the discharging and charging of C_{SM} determines the delays from the starting and ending points, respectively. A smaller C_{SM} can render a faster transient response yet suffers from a larger noise. In many embodiments, the discharging rate of C_{SM} is independent of the amplitude of the Tx signal as it is determined by the current source generated from a bandgap reference block. The recovered timing signal can then be sharpened by a following buffer 330, as shown in FIG. 3 in accordance with an embodiment of the invention. In certain embodiments, the buffer only causes a sub-ns delay. Although FIG. 3 illustrates a particular circuit architecture of a demodulator, any of a variety of circuit architectures may be utilized as appropriate to the requirements of specific applications in accordance with embodiments of the invention.

### Output Voltage Regulator

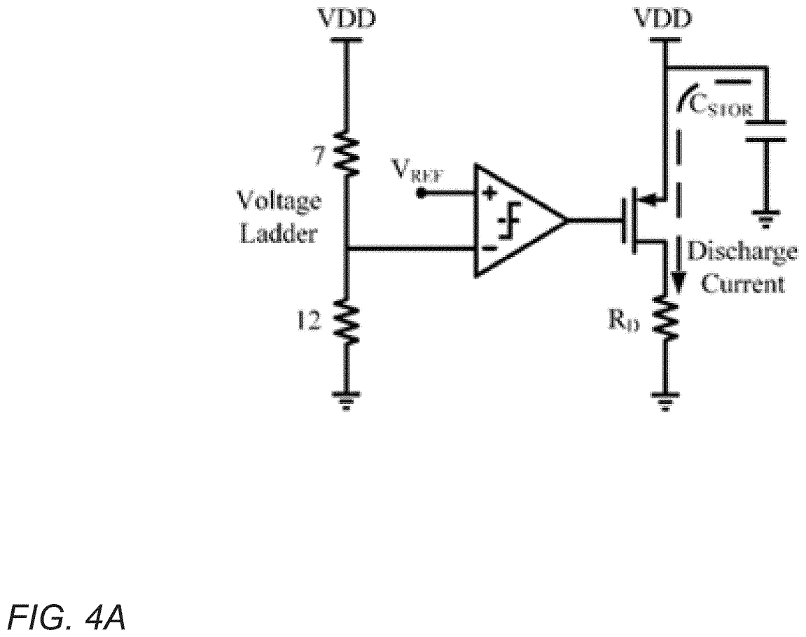

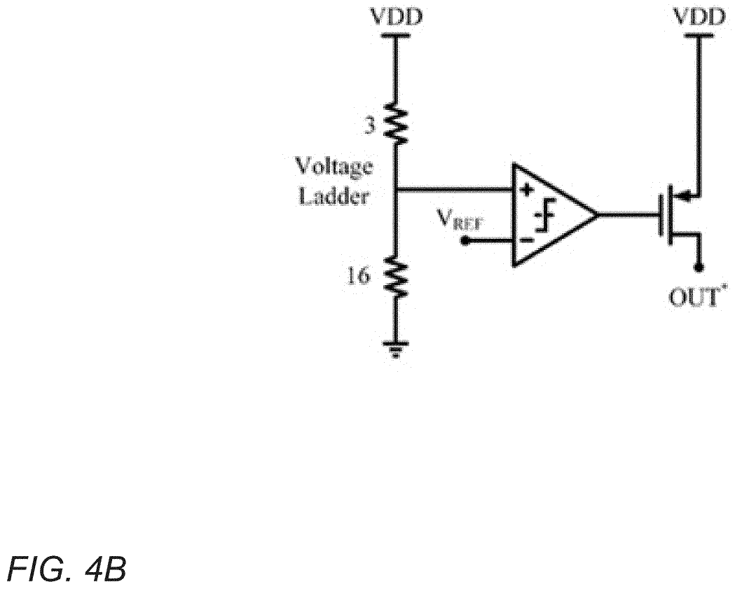

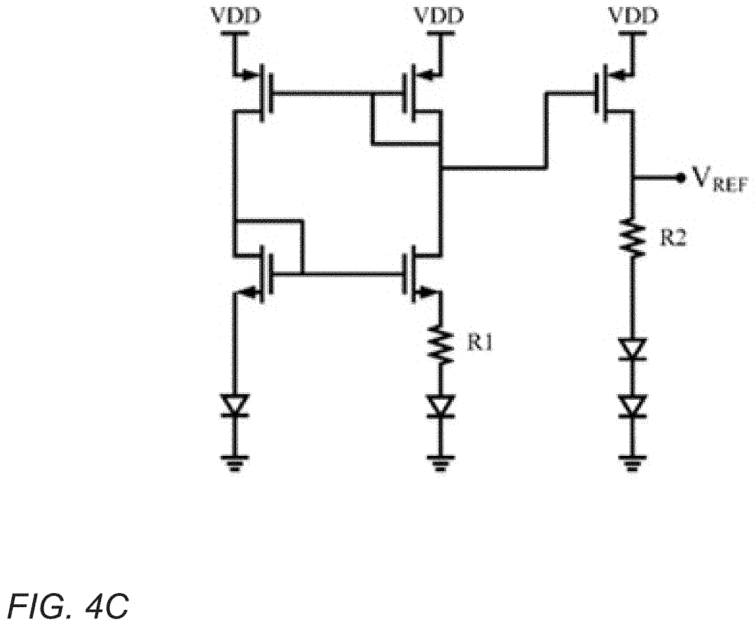

In several embodiments, fractions of VDD can be compared with a constant voltage reference, V_{REF}, so that the amplitude can be regulated within a specific range. Circuits illustrated in FIG. 4A and FIG. 4B in accordance with an embodiment of the invention can determine the high and low bars, respectively. When the supply voltage exceeds 19/12 of V_{REF}, a discharge current path can be enabled through a 65 kΩ resistor, RD, which can rapidly discharge the incident power. On the contrary, in several embodiments, when the amplitude is lower than 19/16 of V_{REF}, OUT* node turns high, which disables the demodulator illustrated in FIG. 3 in accordance with an embodiment of the invention. A bandgap voltage reference circuit in accordance with an embodiment of the invention is shown in FIG. 4C. By tuning R1 and R2, V_{REF} can be designed to be 2.3 V, which can regulate the stimulation amplitude between 2.7 V and 3.6 V. This regulation scheme may eliminate the LDOs which may turn to be the most static power-consuming block in IMDs. The voltage ladder can be further customized to render a narrower window. In certain embodiments, in the actual operation, an excessive Tx power tends to generate pulses with the maximum amplitude. Although FIG. 4A, FIG. 4B and FIG. 4C each illustrate a particular circuit architecture of an output voltage regulator, any of a variety of circuit architectures may be utilized as appropriate to the requirements of specific applications in accordance with embodiments of the invention.

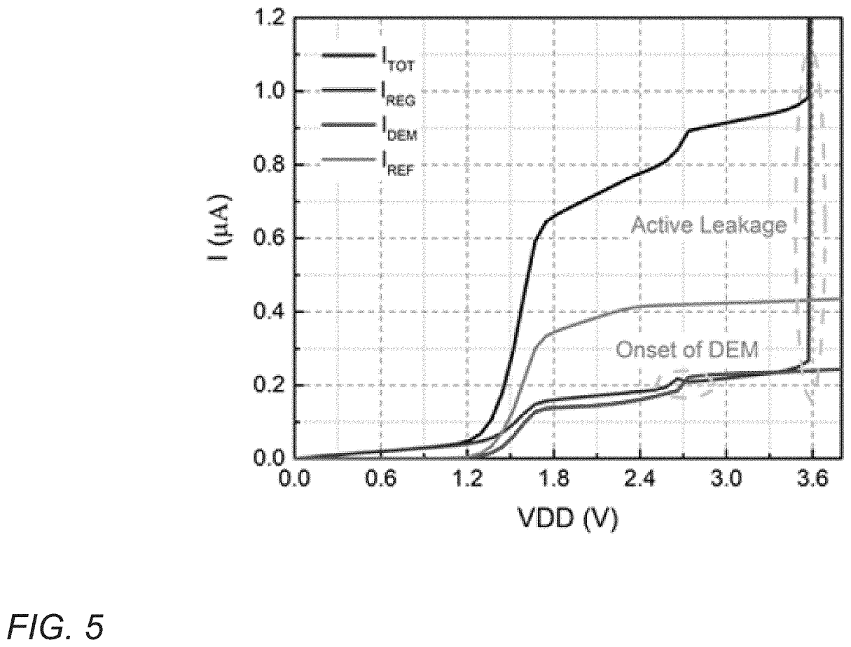

A current consumption of individual blocks is simulated as shown in FIG. 5 in accordance with an embodiment of the invention. With the onset of the demodulator at around 2.7 V, the total current consumption of the IC, I_{TOT}, features a rapid rise (due to the increase of IDEM). When the supply voltage reaches 3.6 V, the leakage path may rapidly discharge the incident power. Below that, the maximum I_{TOT} can be around 950 nA.

### Energy-Harvesting Frontend

In many embodiments, modeling the input impedance of a rectifier as paralleled R and C can provide an intuitive insight into the rectifier design for a resonant coupling system. In the subthreshold region, the input impedance of the rectifier may be dominated by the gate capacitances of the MOS transistors. On the contrary, in several embodiments, as the input voltage swing increases, transistors conduct more current so that the input of the rectifier becomes more resistive.

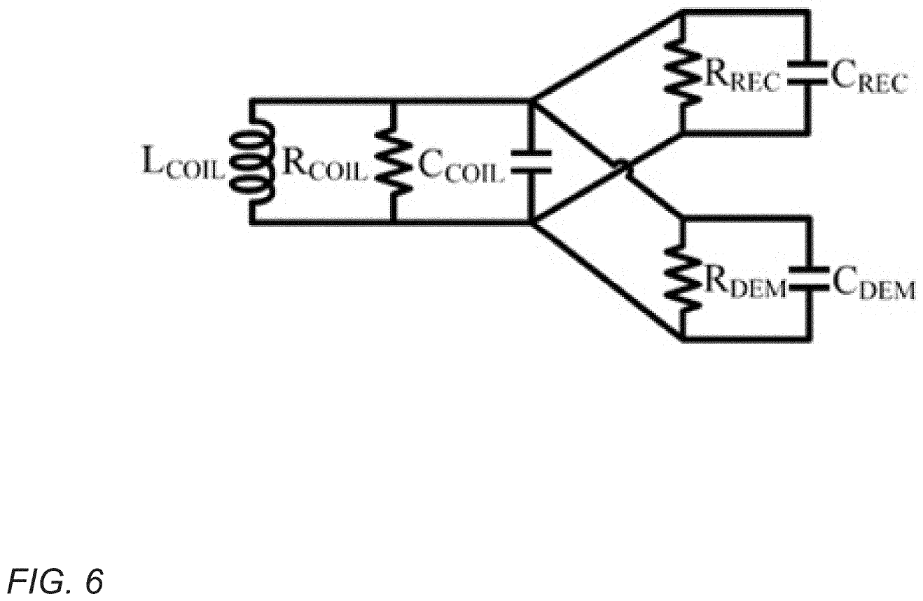

A frontend resonator that includes an Rx coil, rectifier, and demodulator in accordance with an embodiment of the invention is illustrated in FIG. 6. In many embodiments, the Rx coil can be modeled as the parallel configuration of the inductance, L_{COIL}, the loss resistance, R_{COIL}, and the parasitic capacitance, C_{COIL}. In many embodiments, R_{REC} and C_{REC} may represent the input resistance and capacitance of the rectifier, respectively. Similarly, R_{DEM} and C_{DEM} may model the input characteristics of the demodulator. However, in several embodiments, as R_{DEM} and C_{DEM} are simulated to be 1.2 MΩ and 4.7 fF, respectively, they can be omitted. Although FIG. 6 illustrates a particular circuit architecture of an energy-harvesting frontend resonator, any of a variety of circuit architectures may be utilized as appropriate to the requirements of specific applications in accordance with embodiments of the invention.

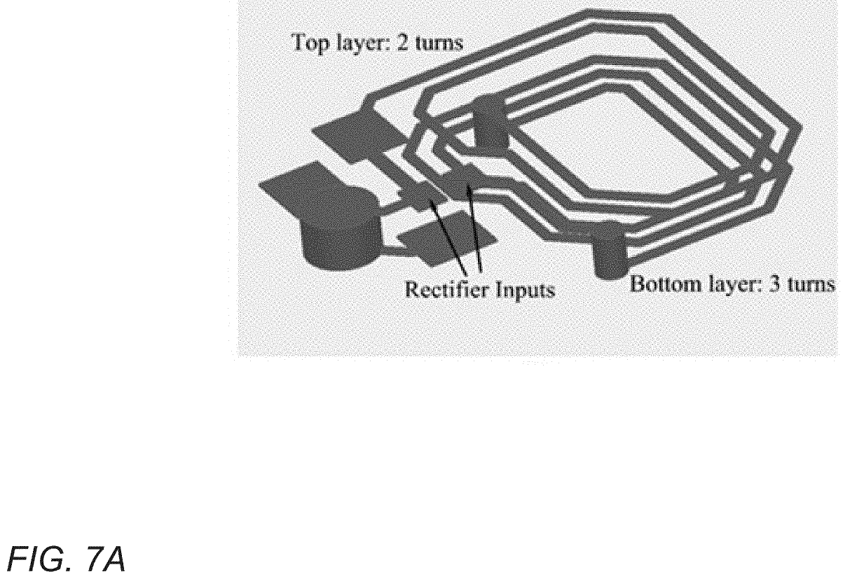

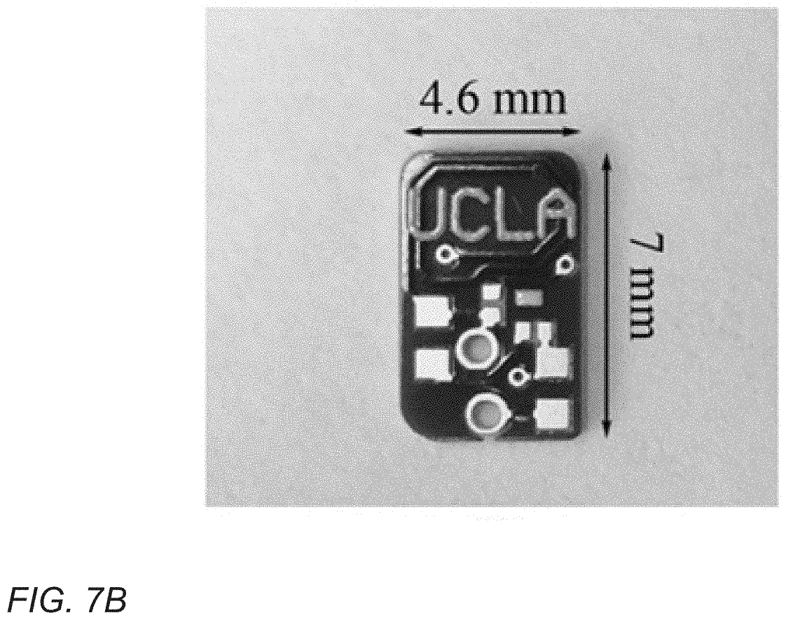

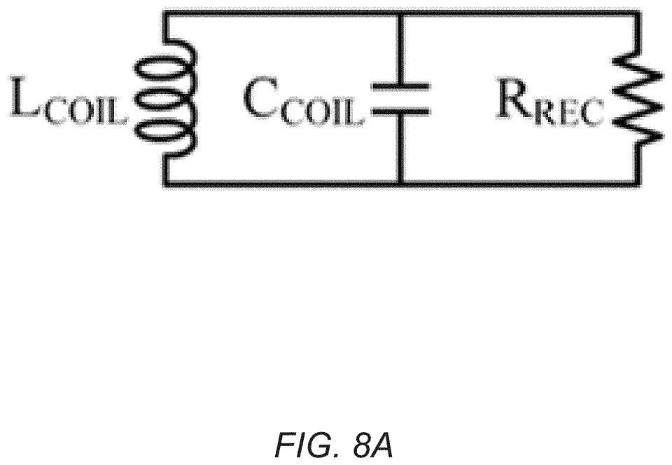

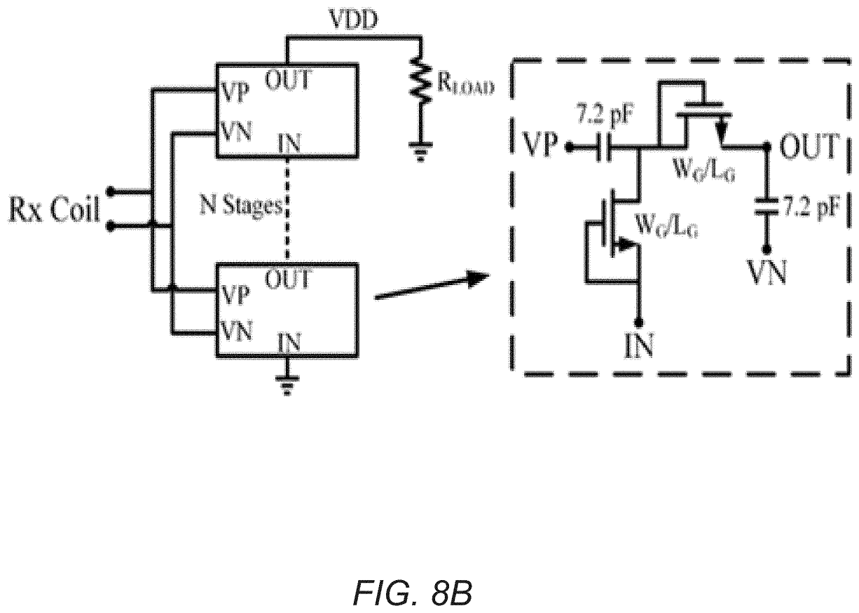

In many embodiments, the Rx coil may dominantly determine the resonant frequency of this resonator. FIG. 7 shows a 3D model and an as-fabricated picture of an Rx coil in accordance with an embodiment of the invention. In certain embodiments, it may reside on 0.5 mm thick Rogers 4350 B substrate and feature a five-turn design with two and three turns on the top and bottom layers, respectively. In several embodiments, the size of the Rx coil can be 4.5 mm×3.6 mm. L_{COIL} can be simulated to be 94.9 nH taking account of all connected traces. As simulations indicate C_{COIL} and R_{COIL} to be an order of magnitude larger than C_{REC} and R_{REC}, respectively, the frontend resonator can be further simplified as illustrated in FIG. 8A in accordance with an embodiment of the invention. The circuit schematic of a Dickson rectifier in accordance with several embodiments is illustrated in FIG. 8B. In certain embodiments, zero-threshold transistors can be used to improve the conversion efficiency. Although FIG. 7 illustrates a particular 3D model of an Rx coil, any of a variety of models may be utilized as appropriate to the requirements of specific applications in accordance with embodiments of the invention. Furthermore, although FIG. 8 illustrates a particular circuit architecture of an energy-harvesting frontend resonator and a Dickson rectifier, any of a variety of circuit architectures may be utilized as appropriate to the requirements of specific applications in accordance with embodiments of the invention.

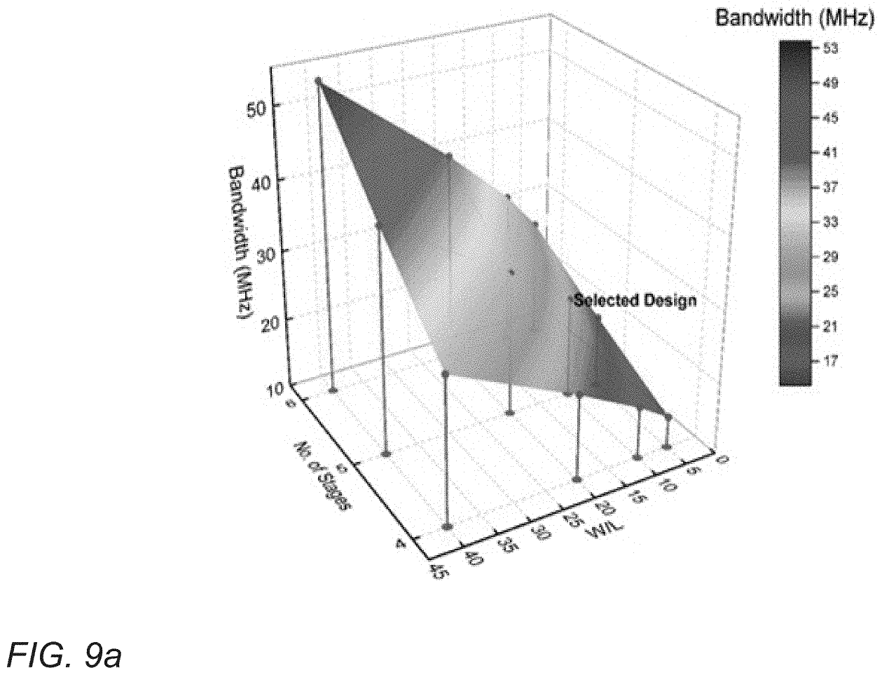

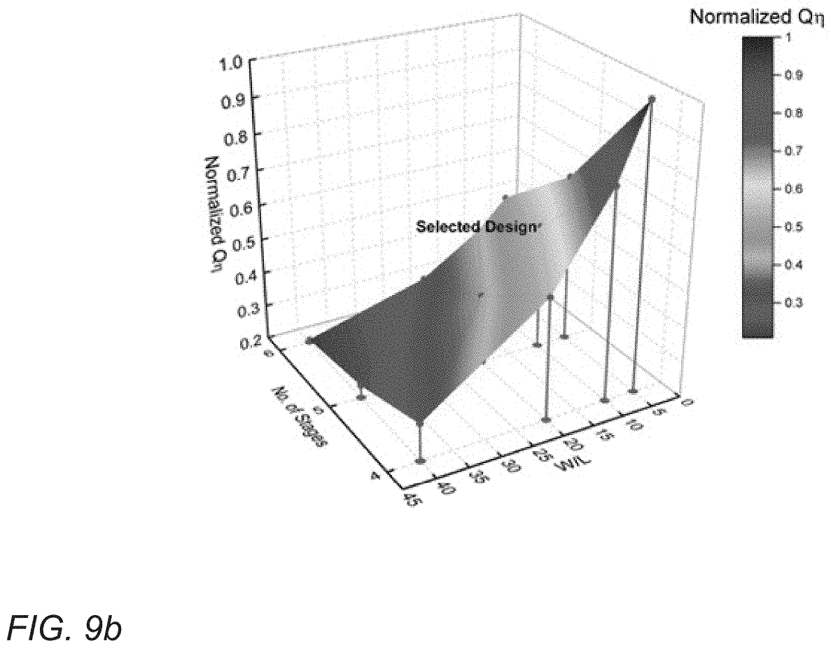

In many embodiments, the design of the rectifier may focus on the tradeoff between the reception sensitivity and bandwidth. Assuming an I_{LOAD} of 5 μA, W_{G}/L_{G} ranging from 2.5 μm/0.5 μm to 20 μm/0.5 μm and the number of stages from 4 to 6 generate different reception bandwidths and sensitivities as shown in FIG. 9 in accordance with an embodiment of the invention. Configurations with more stages and larger W_{G}/L_{G} may render a larger 3 dB-bandwidth of the frontend resonator that can accommodate larger dielectric medium variations, as illustrated in FIG. 9A and FIG. 9B in accordance with an embodiment of the invention. On the contrary, the fewer stages and the smaller W_{G}/L_{G} may lead to a higher reception sensitivity primarily owing to the increased quality factor, Q, as illustrated in FIG. 9B in accordance with an embodiment of the invention. In many embodiments, the reception sensitivity may be compared as the multiplication of Q and the intrinsic conversion efficiency, η, of the rectifier. In many embodiments, a selected design (e.g., W_{G}/L_{G}=5 μm/0.5 μm, N=5) renders a 24 MHz 3 dB-bandwidth and an inherent conversion efficiency of 53% for the rectifier.

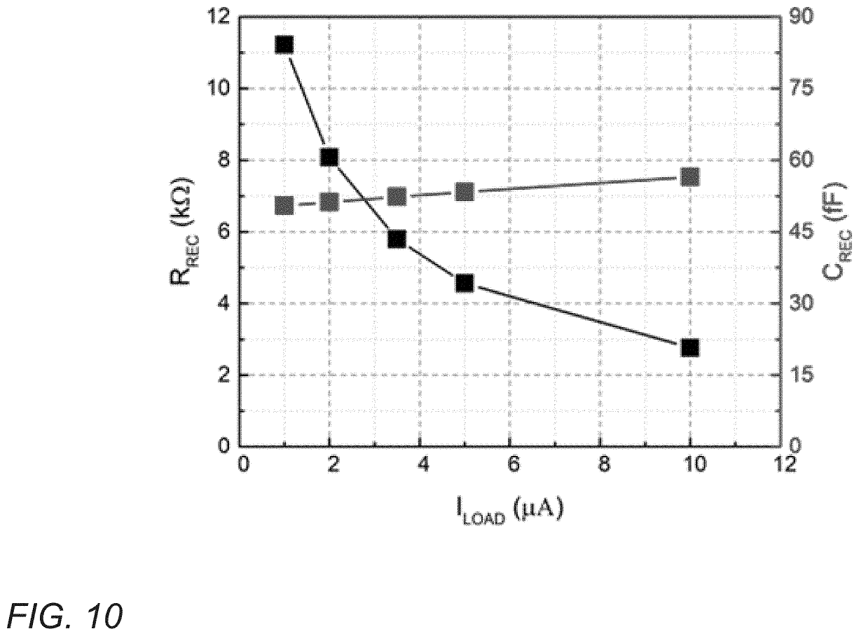

In many embodiments, a selected rectifier design is further simulated to investigate the impacts of I_{LOAD} variations. In certain embodiments, with I_{LOAD} varying from 1 μA to 10 μA, C_{REC} may be remarkably stable at around 50 fF, which verifies the stability of the resonant frequency of the energy-harvesting frontend across a wide range of stimulation loads. On the other hand, R_{REC} may decrease with I_{LOAD}, which indicates an increased reception sensitivity for a lighter load. A simulated dependence of R_{REC} and C_{R}EC on I_{LOAD} is demonstrated in FIG. 10 in accordance with an embodiment of the invention.

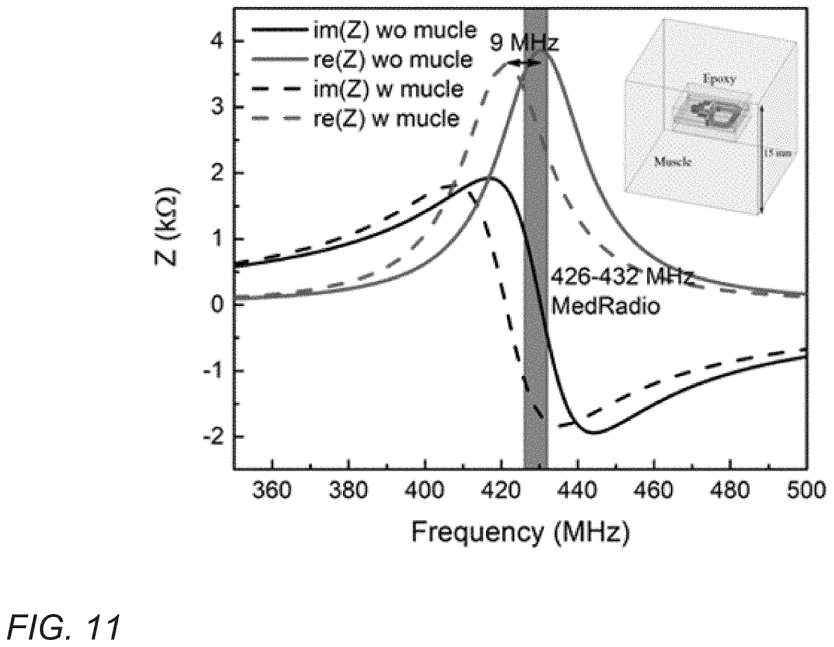

In many embodiments, an IPG assembly can be encapsulated with epoxy. Therefore, the frontend resonator can be simulated within a 3 mm thick epoxy and inside a 1.5 cm muscle cubic to provide an insight into the potential impacts of the dielectric medium variations. In several embodiments, the simulation can be performed with ANSYS and the result shows that the muscle tissue causes a 9 MHz downward drift of the resonant frequency as shown in FIG. 11 in accordance with an embodiment of the invention. In many embodiments, the selected rectifier design succeeds in covering this drift within the 3-dB bandwidth.

FIG. 12 summarizes a procedure for the co-design of the Rx coil and the rectifier targeting a specific MedRadio band in accordance with an embodiment of the invention. In several embodiments, the Rx coil can play a dominant role in determining the resonant frequency. The rectifier can reach the compromise between the reception sensitivity and bandwidth according to the specific load requirement. In several embodiments, this process may need several iterations of optimization to ensure a certain loaded resonant frequency. Although FIG. 12 illustrates a particular co-design procedure for an Rx coil and rectifier, any of a variety of co-design procedures may be utilized as appropriate to the requirements of specific applications in accordance with embodiments of the invention.

### Measurement Results

### Fabrication

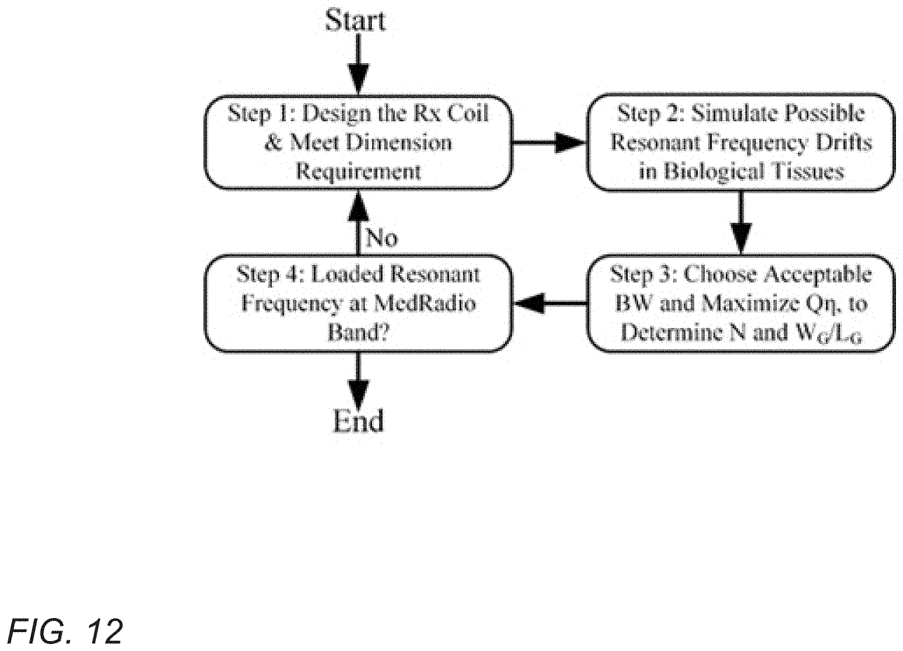

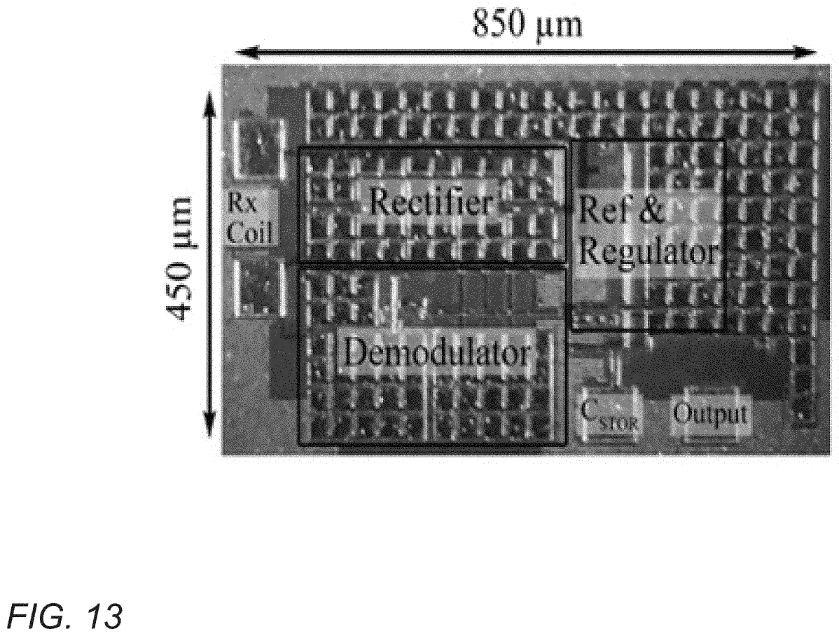

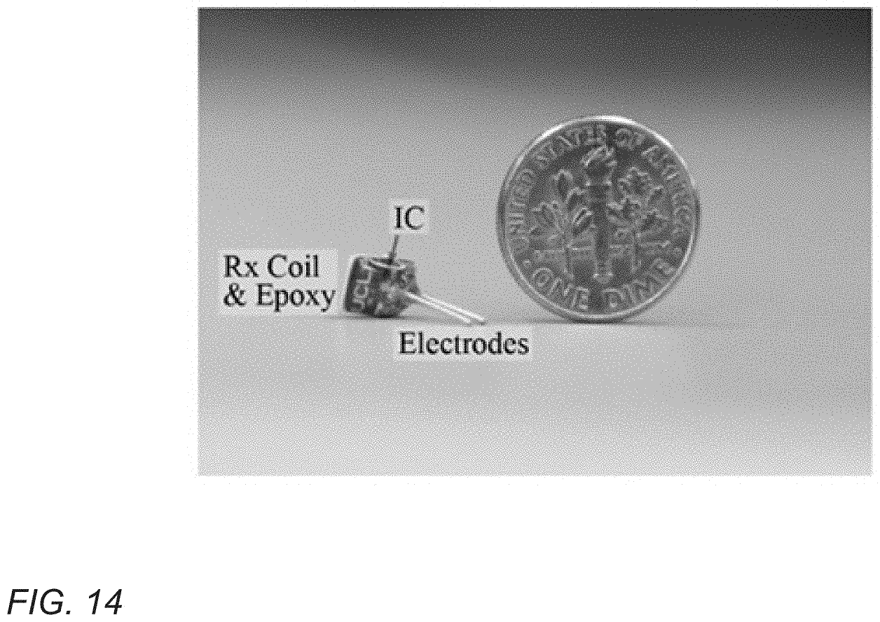

In many embodiments, an IC can be fabricated in TSMC 180 nm CMOS process with a pad-included area of 850 μm×450 μm, as shown in FIG. 13 in accordance with an embodiment of the invention. A picture of an IPG assembly in accordance with an embodiment of the invention is shown in FIG. 14. In certain embodiments, epoxy (e.g., Gorilla 4200101) can be used to encapsulate the assembly and AWG 22 aluminum plated copper wire of about 5 mm can be utilized as the electrodes for simplicity. Although FIG. 13 illustrates an architecture of an IC, any of a variety of architectures may be utilized as appropriate to the requirements of specific applications in accordance with embodiments of the invention.

### Tx Coil

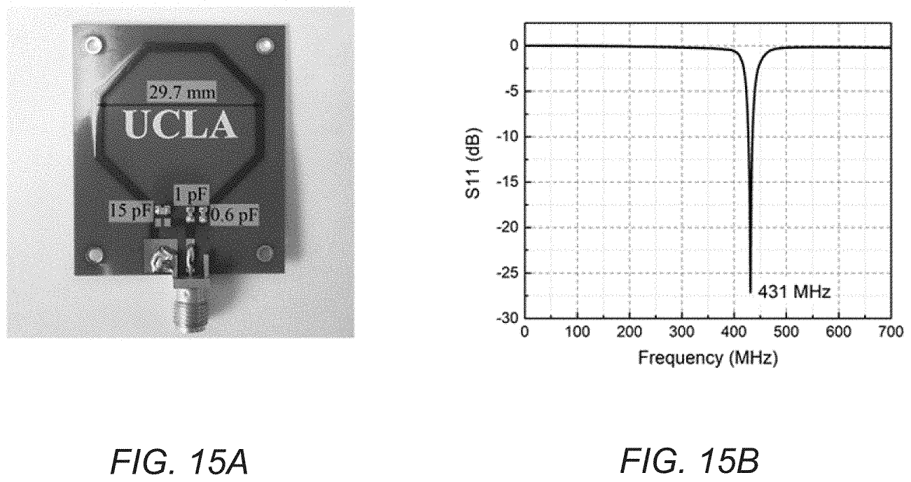

In many embodiments, the Tx coil features a single-turn design and can be implemented on an FR4 substrate, as shown in FIG. 15A in accordance with an embodiment of the invention. In several embodiments, the diameter and trace width can be 29.7 mm and 1.52 mm, respectively. In many embodiments, an L-matching section ensures the impedance matching at 431 MHz as shown in the S11 measurement, as illustrated in FIG. 15B in accordance with an embodiment of the invention. Although FIG. 15 illustrates a particular circuit architecture of a Tx coil, any of a variety of circuit architectures may be utilized as appropriate to the requirements of specific applications in accordance with embodiments of the invention.

### IPG Output

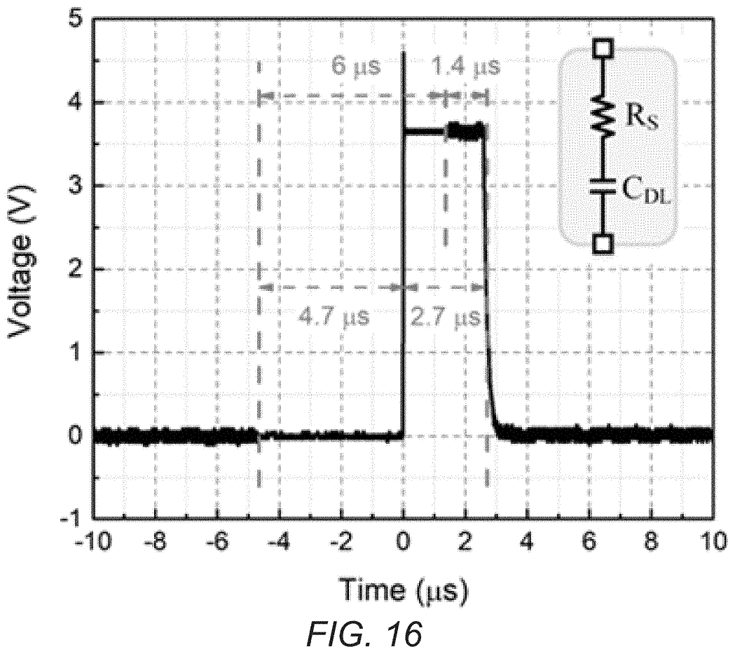

In many embodiments, the electrode impedance can be modeled as a series combination of the tissue/solution resistance, R_{S}, and the double-layer capacitance, C_{DL}, according to works as shown in the inset of FIG. 16 in accordance with an embodiment of the invention. In several embodiments, two electrodes may be immersed in the phosphate buffered solution by approximately 5 mm. RS and CDL can then be characterized to be 1.2 kΩ and 0.6 μF, respectively, with the Stanford Research System SR720 LCR Meter.

In several embodiments, due to the availability of the discrete components, R_{S} of 1.15 kΩ and C_{DL} of 0.6 μF in series may be used as the load of the IPG. In several embodiments, a 6 μs notch may be first applied to the Tx signal, which triggered the output pulse as shown in FIG. 16 in accordance with an embodiment of the invention. In several embodiments, the monophasic waveform has 4.7 μs and 1.4 μs delays compared to the starting and ending points of the notch, respectively. Therefore, the duration of the triggered stimulation can be 3.3 μs shorter than that of the notch. The spike at the onset of the pulse may be an artifact due to parasitic effects of the connection wire.

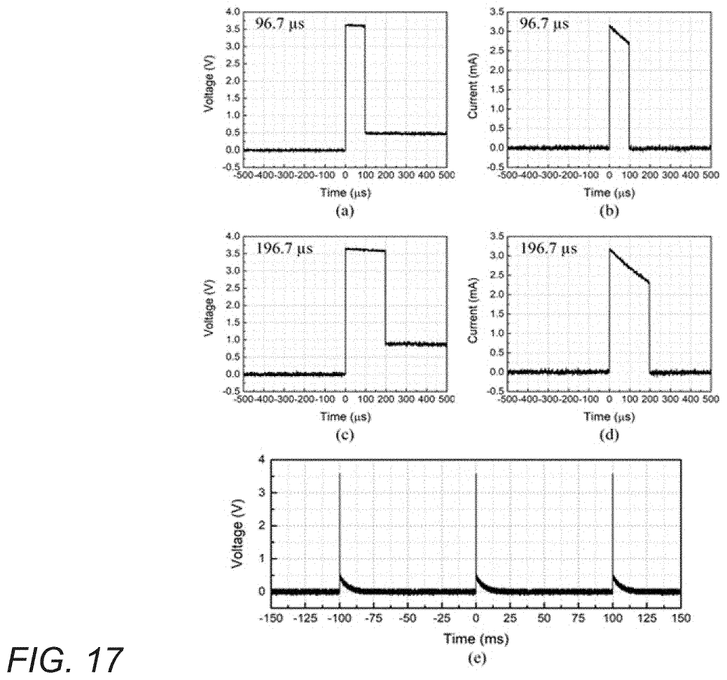

A voltage and corresponding current waveforms for the 96.7 μs and the 196.7 μs pulses are shown in FIG. 17 in accordance with an embodiment of the invention. The injected charge may be temporarily accumulated on C_{DL} so that there appears a post-pulse voltage buildup. In several embodiments, the voltage buildup should not exceed the water delamination window, typically about 1.4 V. In many embodiments, according to this constraint, the pulse width should be kept below 300 μs. The current can be obtained by recording the voltage over the R_{S}, which features an exponentially decaying waveform with the peak of approximately 3.2 mA. In many embodiments, a more comprehensive electrode model may include a charge transfer resistance, R_{CT}, in parallel with C_{DL}, which rapidly discharges the post-pulse potential in saline/tissue. In a typical case, R_{CT} can be around ten times as large as R_{S}. With such R_{CT} of 11 kΩ, the output voltage waveform over multiple cycles is demonstrated in FIG. 17E in accordance with an embodiment of the invention.

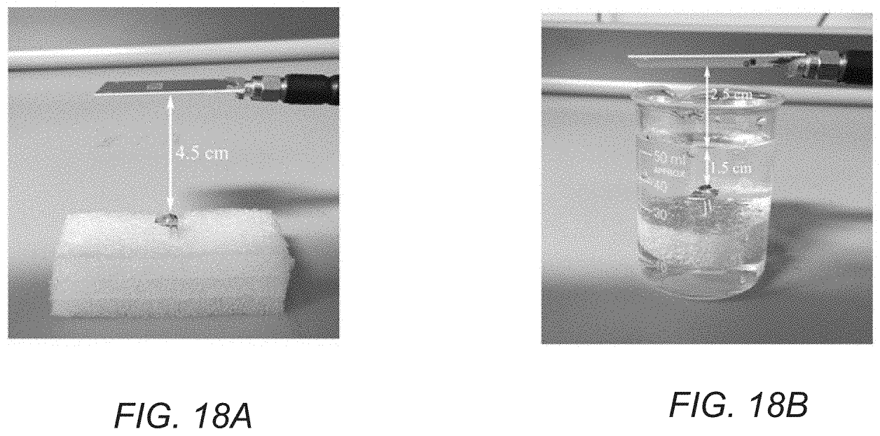

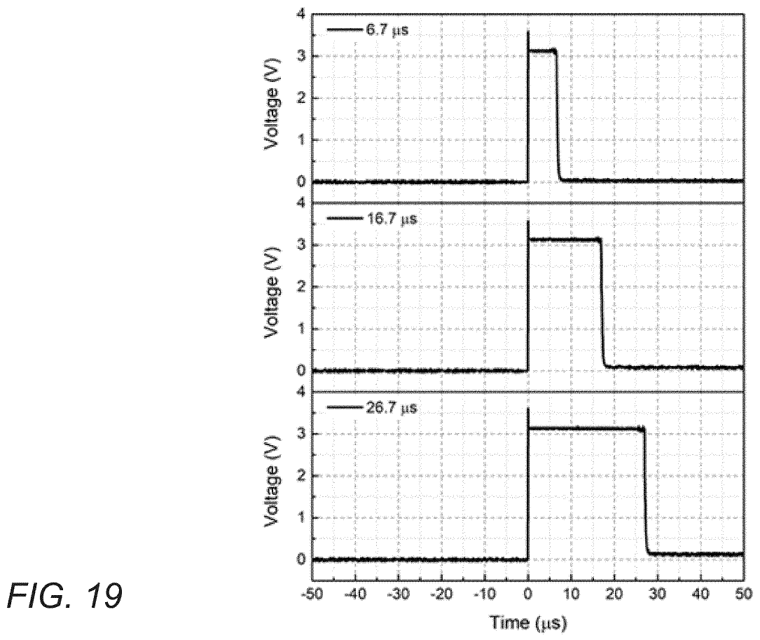

In many embodiments, an LED can be optionally included at the output of the IPG to indicate the occurrence of the output stimulation. In several embodiments, a green LED (e.g., APT1608LZGCK, Kingbright) can be used. In many embodiments, an IPG may be first tested in the air with the Tx power of 1 W. It shows the maximum operating distance of 4.5 cm, as illustrated in FIG. 18A in accordance with an embodiment of the invention. In several embodiments, the device can then be immersed in fresh water (εr=80) at a 1.5 cm depth, as the dielectric constant mimics that of the body. The Tx coil may operate the IPG at 2.5 cm above the water surface with a total distance of 4 cm as illustrated in FIG. 18B in accordance with an embodiment of the invention. The LED may regulate the amplitude of the output pulse at 3.1 V. 6.7 μs, Waveforms of 16.7 μs, and 26.7 μs pulses respectively triggered by 10 μs, 20 μs, and 30 μs notches are demonstrated in FIG. 19 in accordance with an embodiment of the invention.

### Animal Experiment

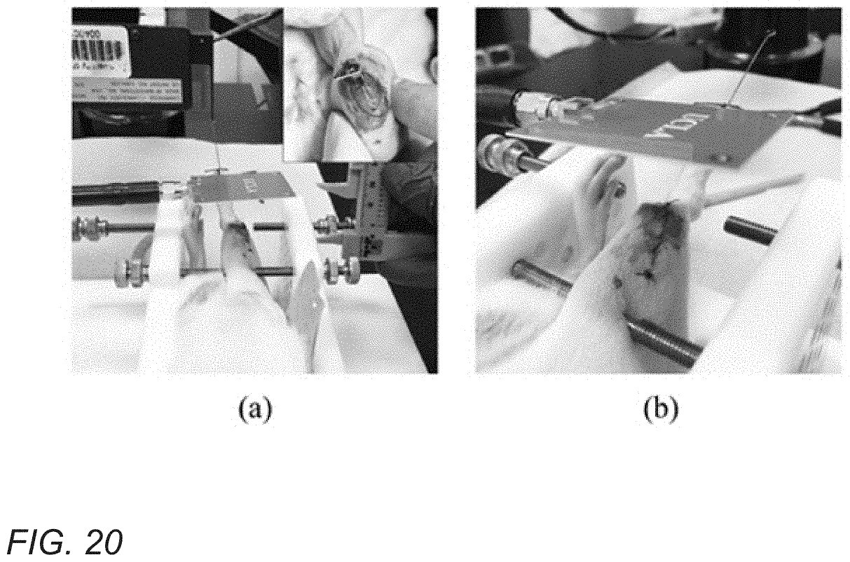

Selective activation of specific muscles with a miniaturized implantable stimulator has been shown to correct foot drops. An in vivo experiment has been performed to test the use of the IPG in neuromuscular stimulations. In the experiment, a rat was initially anesthetized with urethane anesthesia (1.2 g/kg) administered subcutaneously. An IPG device (w LED) was inserted into the muscle (Tibialis Anterior) belly with the two electrodes about 2 mm apart. The device was secured in place with 4-0 Ethilon suture, as shown in the inset of FIG. 20A. The Tx coil was placed 3 cm above the hind limb with the source power of 1 W at 430 MHz. The connective tissue and skin were sewn covering the device. The rat was placed on the back with the knee joint secured using metallic screws. The toes were directly connected to a force transducer to measure isometric contractions, as shown in FIG. 20A. FIG. 20B displays a closer view of the implantation site. The force transducer was then connected to a DAQ that digitizes and records the data (sampling frequency=10 kHz). All procedures were in accordance with the National Institute of Health Guide for the Care and Use of Laboratory Animals and were approved by the Animal Research Committee at UCLA.

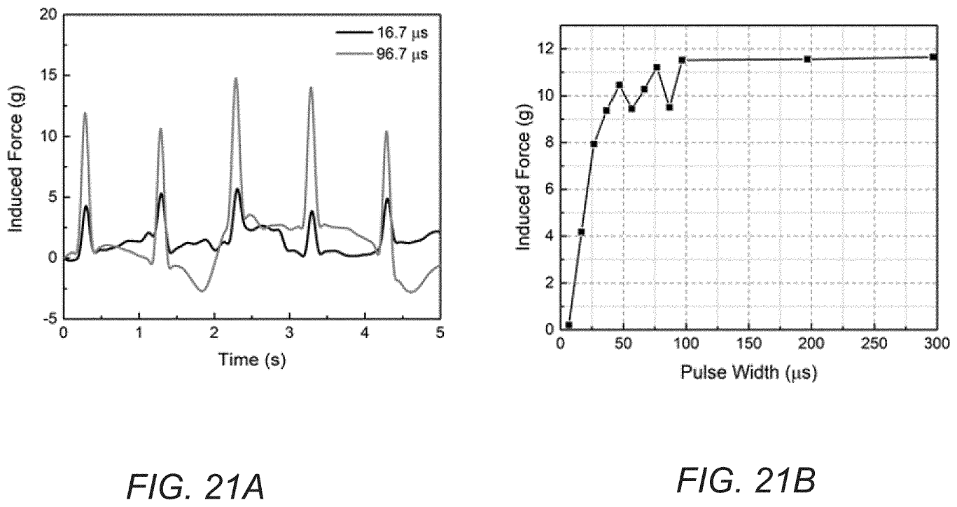

The stimulation intensity was varied with each pulse width repeated at least 10 times to ensure reproducibility. The pulse rate was fixed at 1 Hz in this experiment. A minimum of 2 min break was given between two pulse width cycles to account for muscle fatigue. Transient recordings of the induced force with 16.7 μs and 96.7 μs pulses are demonstrated in FIG. 21A. During the response to a stimulus, the motor output demonstrates minor variations due to the inherent variability in the nervous system. In addition, with each isometric contraction, the foot of the animal may be deflected, thus affecting the baseline force. The dependence of the induced force on the pulse width is shown in FIG. 21B. Peak to baseline force was calculated and averaged for 10 pulses at each pulse width. The force monotonically increases until a plateau for pulse widths above 100 μs. This non-linear relationship observed as a recruitment curve is consistent with that observed previously. The recruitment curve is a common strategy used for identifying the appropriate stimulation parameters.

### Calculation of Charge Delivering

In many embodiments, calculation of the injected amount of charge provides an insight into the proper design of the electrodes for voltage-controlled IPGs. Assuming the voltage buildup on C_{BCK} to be V_{X} (V_{X} typically much smaller than VDD), the delivered amount of charge with each stimulation equals

ΔQ_{ch}=(VDD−V_{X})(1−e^{-T}^{_{Pulse}}^{/R}^{_{S}}^{C}^{_{DL}})C_{DL} (1)

Where T_{Pulse} presents the pulse width. The amplitude of the injected current exponentially decays as determined by the time constant according to the electrode model shown in FIG. 16 in accordance with an embodiment of the invention. In the animal experiment, since the pulse amplitude is regulated by the LED at around 3 V, 16.7 μs and 96.7 μs pulses deliver approximately 0.04 μC and 0.23 μC charge, respectively. Multiplying ΔQ_{ch} by the pulse rate, F_{Pulse}, the delivered amount of charge in each second equals

Q_{ch}=F_{Pulse}ΔQ_{ch} (2)

Note that Q_{ch} is accumulated on C_{BCK}. Therefore, the passive discharging path should suffice the following relationship,

V_{X}/R_{DIS}>Q_{ch} (3)

A smaller R_{DIS} in the assembly will ensure a smaller V_{X} that does not evidently hamper the intensity of each stimulation. Many embodiments aim for a μW-level simulation load, R_{DIS} may be selected to be 200 kΩ to ensure a minimum V_{X}. In many embodiments, C_{BCK} can be 47 μF. A relatively large C_{BCK} may help to stabilize V_{X}.

### SAR Evaluation

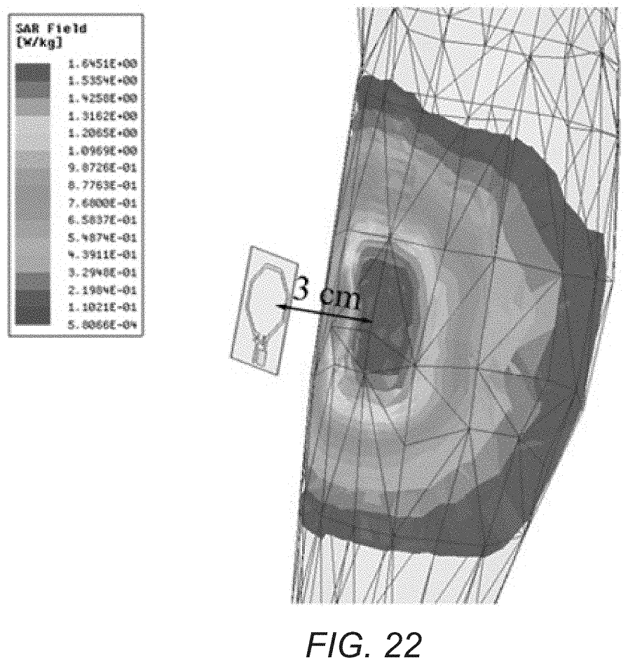

An SAR evaluation may be performed in ANSYS. In many embodiments, placing the Tx coil at a 3 cm distance from the human leg model, the simulated 10-g averaged SAR features the maximum value of 1.645 W/kg with the Tx power of 1 W, as shown in FIG. 22 in accordance with an embodiment of the invention. In many embodiments, the SAR may be well below the restrictions for localized exposure according to IEEE Std C95.1-2005, i.e., the lower tier of 2 W/kg used for general public and the higher tier of 10 W/kg used for controlled environments, e.g. medical implant use.

### Comparisons

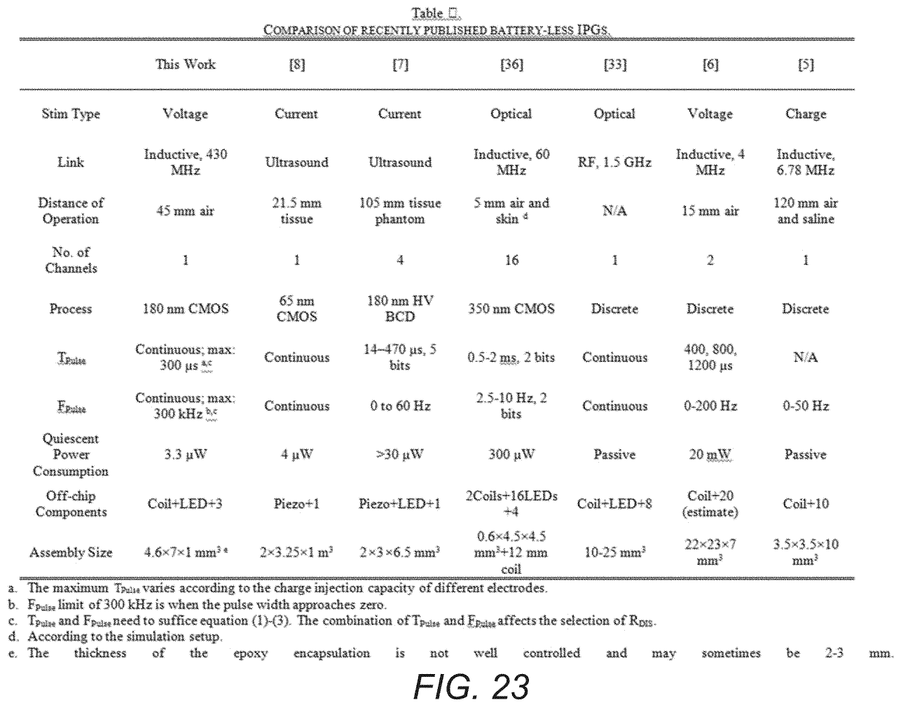

A comparison with recently published miniaturized IPGs is presented in the table illustrated in FIG. 23. Due to the elimination of the coil, ultrasound-based IPGs tend to have smaller form factors. However, their operation typically requires the use of the ultrasound gel. In addition, concerns were with its propagation through air-filled viscera such as the lung and bowel, and obstructions such as bones. Passive circuits have also been investigated to realize energy-efficient IPGs. However, they require sudden bursts of the Tx power, which are more prone to violate the SAR regulations. To achieve a high reception sensitivity, many embodiments of the IPG consume one of the lowest static powers among active circuitry-based works. The use of MedRadio-band may contribute to the miniaturized form factor of the implant. In many embodiments, replacing the discrete components currently in 0603 SMD packages to 0201 ones can further reduce the overall size by a large portion.

Although specific implementations for an IPG are discussed above with respect to FIGS. 1-23, any of a variety of implementations utilizing the above discussed techniques can be utilized for an IPG in accordance with embodiments of the invention. While the above description contains many specific embodiments of the invention, these should not be construed as limitations on the scope of the invention, but rather as an example of one embodiment thereof. It is therefore to be understood that the present invention may be practiced otherwise than specifically described, without departing from the scope and spirit of the present invention. Thus, embodiments of the present invention should be considered in all respects as illustrative and not restrictive.

What is claimed is:

1. A wirelessly powered stimulator, comprising:
an implantable pulse generator (IPG), comprising:
an Rx antenna that receives a radio frequency (RF) signal from an external Tx antenna;
a rectifier;
an energy storage capacitor C_{STOR}, wherein the RF signal coupled to the Rx antenna is rectified by the rectifier to generate VDD and charges the C_{STOR};
a demodulator;
an output voltage regulator that generates a stable voltage to activate the demodulator; and
wherein the demodulator outputs a stimulation that releases the energy stored in the C_{STOR} on an electrode based on detecting amplitude modulation in the received RF signal;
a Tx antenna that generates the RF signal that wirelessly powers the IPG and that controls timing of output stimulations of the IPG, wherein amplitude modulation is applied to the RF signal to control the timing of the output stimulations.

2. The wirelessly powered stimulator of claim 1, wherein the IPG further comprises a plurality of reverse bias diodes that release energy from the C_{STOR} when the energy stored reaches an upper level threshold.

3. The wirelessly powered stimulator of claim 1, wherein the Rx antenna is at least one antenna selected from the group consisting of an inductor coil, a resonant coil, a dipole antenna, a monopole antenna, a patch antenna, a bow-tie antenna, a phased-array antenna, and a wire.

4. The wirelessly powered stimulator of claim 1, wherein the C_{STOR} is off-chip.

5. The wirelessly powered stimulator of claim 1, wherein the C_{STOR} is on-chip.

6. The wirelessly powered stimulator of claim 1, wherein the Rx antenna is off-chip.

7. The wirelessly powered stimulator of claim 1, wherein the Rx antenna is on-chip.

8. The wirelessly powered stimulator of claim 1, wherein amplitude modulation comprises detecting at least a threshold percentage reduction in power of the RF signal from the Tx antenna.

9. The wirelessly powered stimulator of claim 1, further comprising a DC-block capacitor, C_{BCK}, that delivers the output stimulations for charge-neutralization.

10. The wirelessly powered stimulator of claim 9, further comprising a discharge resistor, R_{DIS}, that nulls the accumulated charge on the C_{BCK}.

11. The wirelessly powered stimulator of claim 1, wherein the IPG is used for at least one application selected from the group consisting of neural stimulation, heart pacing, defibrillation, bladder stimulation and deep brain stimulation.

12. The wirelessly powered stimulator of claim 2, wherein the output voltage regulator limits an amplitude of output stimulations within a specific range, wherein the output voltage regulator enables the demodulator when a supply voltage exceeds a lower tier; and wherein when the supply voltage exceeds a higher tier, enables a discharge path to rapidly discharge excess incident charge.

13. The wirelessly powered stimulator of claim 1, wherein the amplitude modulation is applied to the RF signal to control at least one of a repetition rate and a duration of the output stimulation in an analog manner.

14. The wirelessly powered stimulator of claim 1, wherein the demodulator replicates a timing of the amplitude modulation applied to the RF signal.

15. The wirelessly powered stimulator of claim 14, wherein the demodulator comprises three source follower replicas with a high end V_{H}, low end V_{L}, and transient envelop V_{ENV} of the RF signal and the V_{ENV} detection branch uses a small capacitor Cₛₘ and V_{H} and V_{L} are extracted on large capacitors with and without the AC input respectively.

16. The wirelessly powered stimulator of claim 15, wherein an average of V_{H} and V_{L}, V_{M}, is obtained using a resistive divider and compared with V_{ENV} to reconstruct the timing of the amplitude modulation.

17. The wirelessly powered stimulator of claim 15, wherein a recovered timing signal is sharpened by a buffer.
